(19) United States
(12) Patent Application Publication
(10) Pub. No.: US 2013/0203863 A1
(43) Pub. Date: Aug. 8, 2013

(54) METHOD FOR PREPARING A COMPOSITION INCLUDING A COMPOUND CONTAINING VANILLIN AND ETHYLVANILLIN, COMPOSITION THUS OBTAINED AND USE THEREOF

(71) Applicants: Jean-Claude Le-Thiesse, Saint-Etienne (FR); Kilani Lamiri, L'arbresle (FR)

(72) Inventors: Jean-Claude Le-Thiesse, Saint-Etienne (FR); Kilani Lamiri, L'arbresle (FR)

(73) Assignee: RHODIA OPERATIONS, Aubervilliers (FR)

(21) Appl. No.: 13/581,276

(22) PCT Filed: Feb. 21, 2011
(86) PCT No.: PCT/EP2011/052534

§ 371 Date: Nov. 1, 2012

(30) Foreign Application Priority Data
Feb. 24, 2010 (FR) 1000747

(51) Int. Cl. A23L 1/226; C11D 3/50; A61Q 13/00; A61K 8/33; A61K 47/08

(52) U.S. Cl. CPC A23L 1/22657; A61K 8/33; A61K 47/08; A61Q 13/00; C11D 3/50
USPC 514/772; 512/21; 510/102; 426/453; 426/538

(57) ABSTRACT

A method for preparing a composition including a compound including vanillin and ethylvanillin is described. Also described, is a method of preparing a composition including a compound including vanillin and ethylvanillin according to a vanillin/ethylvanillin molar ratio of 2. The method can include an operation of co-granulating vanillin and ethylvanillin at a temperature of 50° C. to 57° C. in the form of a powder and in amounts such that the vanillin/ethylvanillin molar ratio is at least equal to 2, and an operation of lowering the temperature of the composition thus obtained to room temperature.

The present invention relates to a process for preparing a composition comprising essentially a compound based on vanillin and ethyl vanillin.

Vanillin or 4-hydroxy-3-methoxybenzaldehyde is a product widely used in many fields of application as a flavoring and/or fragrance.

Thus, vanillin is consumed abundantly in the food and animal-feed industry, but it also has applications in other fields, such as, for example, pharmacy or perfumery. Consequently, it is a product with a high level of consumption.

Vanillin is very often combined with ethyl vanillin or 3-ethoxy-4-hyrdoxybenzaldehyde, since it is known that the presence of a small amount of ethyl vanillin makes it possible to intensify the fragrancing and/or organoleptic properties of vanillin.

Thus, a potential user would like to be provided with a ready-made mixture of vanillin and ethyl vanillin.

The problem that arises is that preparing said mixture by means of a conventional technique of dry mixing of vanillin and ethyl vanillin powders results in the production of a mixture which is very liable to cake. As a result, it is impossible to use such a mixture owing to its presentation, which is not in pulverulent form, and to very great difficulty in solubilizing the mass obtained.

Moreover, prolonged storage leads to a worsening of the caking phenomenon, resulting in the powder setting.

Thus, it is desirable to have available a new presentation in solid form, based on vanillin and ethyl vanillin, which has improved flowability properties and an absence of caking on storage.

The applicant has found, according to French patent application No. 08 05913, that a new compound obtained by co-crystallization of vanillin and ethyl vanillin used in a vanillin/ethyl vanillin molar ratio of 2, exhibits unique properties, in particular with regard to its flowability properties and its lack of caking.

Said compound is in the form of a white powder which has a melting point, measured by differential scanning calorimetry, of 60° C.±2° C., different than that of vanillin and ethyl vanillin, of 81° C.±1° C. and 76° C.±1° C., respectively.

It has its own specific X-ray diffraction spectrum, which is different than that of vanillin and ethyl vanillin.

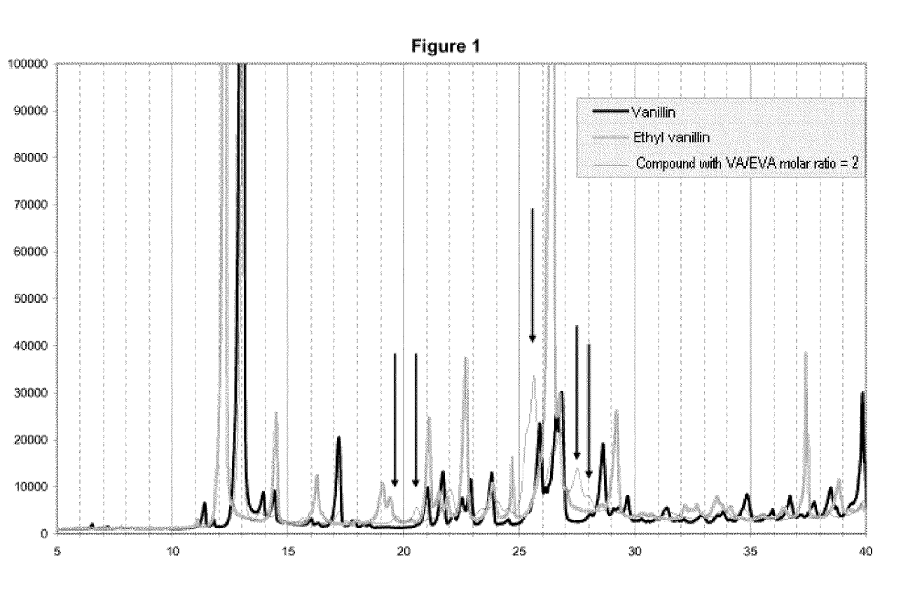

FIG. 1 shows three curves corresponding to the various X-ray diffraction spectra of the new compound of vanillin and ethyl vanillin, of vanillin and of ethyl vanillin.

On the spectrum of the new compound of vanillin and ethyl vanillin, the presence of lines at angles 2θ (°)=20.7-25.6-27.5-28.0 is in particular noted; said lines being absent from the X-ray diffraction spectra of vanillin and of ethyl vanillin.

Another characteristic of said compound is that its X-ray diffraction spectrum does not undergo any significant modification during prolonged storage.

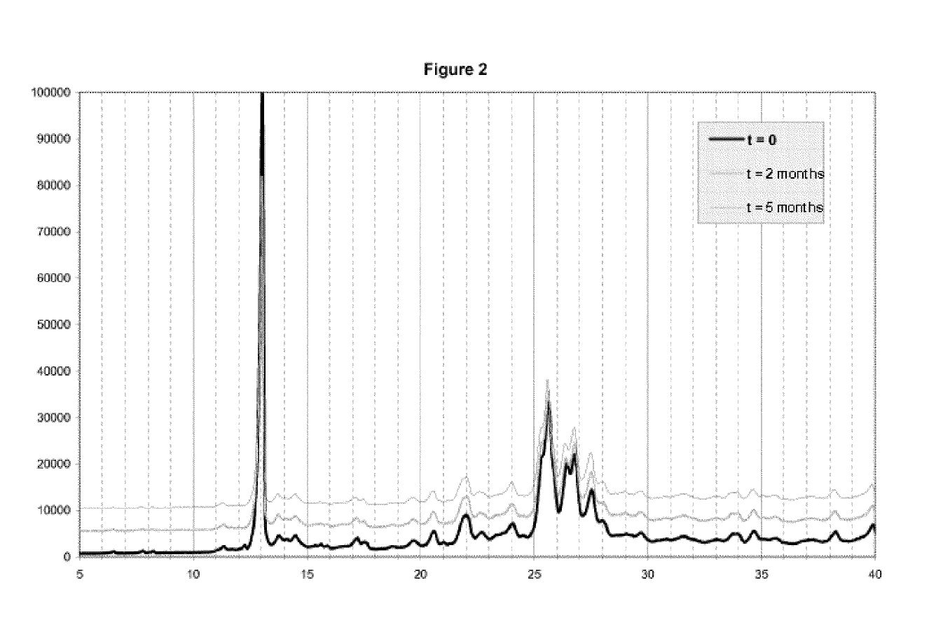

The change in its spectrum was monitored as a function of the storage time at ambient temperature. Over a prolonged storage period (five months), absolutely no modification of the spectrum of the new compound is observed, as demonstrated in FIG. 2.

FIG. 2 shows the change in the X-ray diffraction spectrum of the new compound, as a function of the storage time. It shows three curves corresponding to the various X-ray diffraction spectra of the compound of the invention obtained at time t=0, and then after storage for two months and five months.

The three curves obtained are normally superimposed. In order to be able to distinguish them better, two of these three curves of FIG. 2 have a base line that is intentionally shifted relative to the reference base line, which is the X-ray diffraction spectrum at time t=0. The curve corresponding to the X-ray diffraction spectrum obtained after storage for two months is shifted by 5000 counts/s and that obtained after storage for five months is shifted by 10 000 counts/s.

FIG. 2 demonstrates that there is no change in the compound of the invention after prolonged storage.

An absence of modification of the specific lines of the new compound of vanillin and ethyl vanillin with a vanillin/ethyl vanillin molar ratio of 2 is noted.

Another characteristic of said compound is that it is a compound that is not or very sparingly hygroscopic like vanillin and ethyl vanillin.

The hygroscopicity of said compound is determined by measuring its weight change after having been kept at 40° C. for 1 hour under air at 80% relative humidity.

Said compound adsorbs less than 0.5% by weight of water, and its content is preferably between 0.1 and 0.3% by weight of water. Said compound remains perfectly solid.

Moreover, this compound has good organoleptic properties and it possesses a high aromatic power which is far greater than that of vanillin.

Thus, the compound as defined and which is denoted in the remainder of the text “new compound” has specific properties which are reflected by a reduced ability to cake compared with a composition of vanillin and ethyl vanillin obtained by simple dry mixing.

The particular properties of the compound based on vanillin and ethyl vanillin as previously described are linked to two parameters, namely the molar ratio between the vanillin and the ethyl vanillin and the fact that there is co-crystallization between the vanillin and ethyl vanillin in a specific crystalline form characterized by its melting point and its X-ray diffraction spectrum.

One of the routes for obtaining said compound lies in a process which consists in melting the mixture of vanillin and ethyl vanillin used in a molar ratio of 2, then cooling the molten mixture by reducing the temperature to 50°±1° C., and then maintaining this temperature until the mixture has completely solidified.

The cooling is advantageously carried out in the absence of any stirring.

To this effect, the vanillin and the ethyl vanillin used in a molar ratio of 2 are loaded separately or as a mixture, and the mixture is brought to a temperature which is selected between 60° C. and 90° C. and which is preferably between 70° C. and 80° C.

It is desirable to carry out the preparation of this mixture under an atmosphere of inert gas, which is preferentially nitrogen.

The mixture is kept at the selected temperature until the molten mixture is obtained.

The molten product is transferred into any container, for example a stainless steel tray that will allow easy recovery of the product after solidification. This container is preheated to between 70 and 80° C. before it receives the molten mixture.

In a subsequent step, the molten mixture is cooled to a temperature of 50° C.±1, by controlling the cooling temperature by any known means.

As mentioned previously, the cooling is preferably carried out in the absence of any stirring.

The solidified mixture obtained can then be formed according to various techniques, in particular milling.

This process therefore makes it possible to obtain the new compound of vanillin and ethyl vanillin, but it has the disadvantage of not being readily transposable to the industrial scale since the crystallization of the compound is quite slow. This is because said compound exhibits a supercooling phenomenon, i.e. when the product is molten and it is cooled below its melting point, it crystallizes with difficulty and remains in the liquid state for a long time. The time required for the crystallization is more or less random and it is important to correctly control the crystallization.

Thus, cooling to a temperature of less than 50° C.±1, for example 20° C., makes it possible to accelerate the process of solidification of the molten mixture, but the crystallization is heterogeneous with the coexistence of various crystalline phases, some of which are unstable at ambient temperature or very hygroscopic. This results in considerable caking on storage of a vanillin-ethyl vanillin mixture crystallized under such conditions.

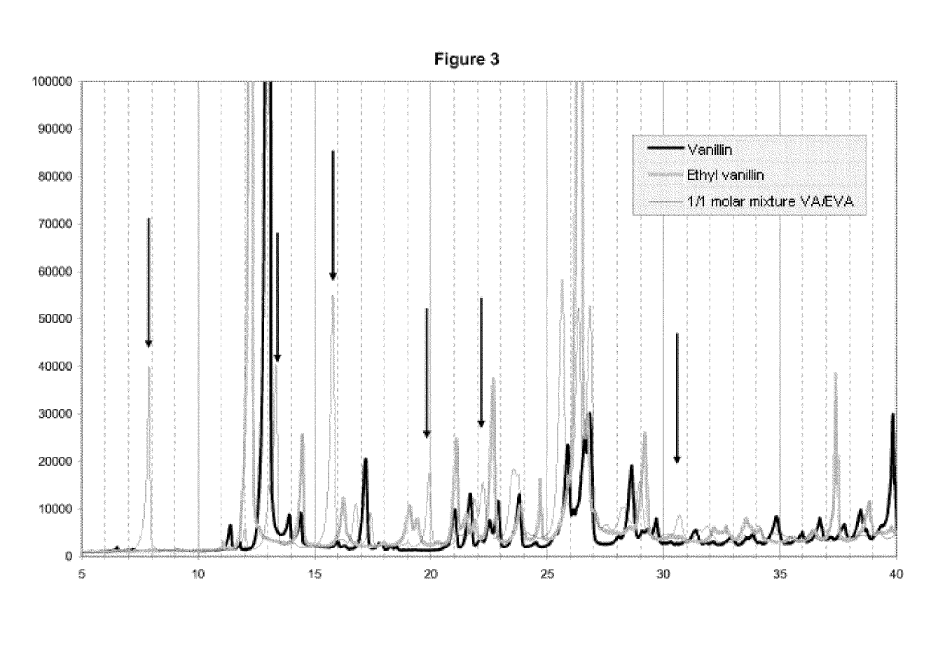

By way of comparative example, in order to illustrate the importance of the vanillin-ethyl vanillin molar ratio and the conditions for crystallization of the molten mixture, FIG. 3 represents the X-ray diffraction spectrum of an equimolar vanillin-ethyl vanillin mixture, melted at 70° C., then crystallized by rapid cooling to 20° C.

This spectrum is different than that of vanillin, than that of ethyl vanillin and than that of the new compound of vanillin and ethyl vanillin with a vanillin/ethyl vanillin molar ratio of 2, with specific lines in particular at angles 2θ (°)=7.9-13.4-15.8-19.9-22.2-30.7.

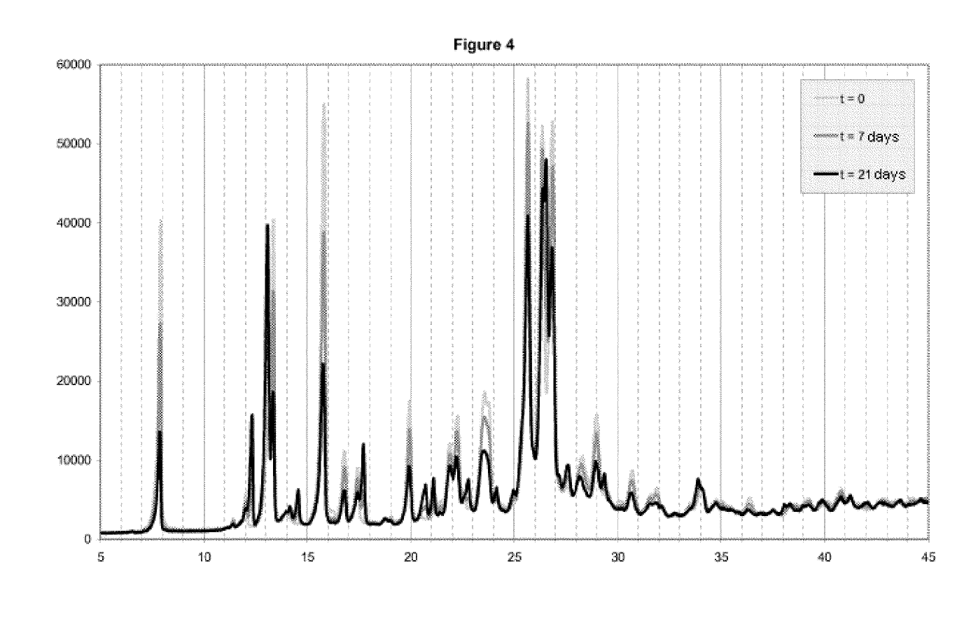

FIG. 4 shows the change in this spectrum over a storage period of three weeks at 22° C., proving that the phases thus crystallized are unstable and change rapidly while causing caking of the product.

This product has a melting point of 48° C.±1 and is found to be very hygroscopic: over the course of 1 hour at 40° C. and under air at 80% relative humidity, it adsorbs more than 4% of water by weight and becomes deliquescent.

Its properties are therefore very different than those of the new compound as previously described and do not make it possible to solve the caking problems posed by vanillin-ethyl vanillin mixtures.

The objective of the present invention is to provide a process transposable to the industrial scale, which makes it possible to obtain essentially the new compound of vanillin and ethyl vanillin with a vanillin/ethyl vanillin molar ratio of 2.

Another objective of the invention is that it results in a composition comprising same, which has the improved properties as mentioned above.

There has now been found, and it is this which constitutes the subject of the present invention, a process for preparing a composition comprising essentially a compound based on vanillin and ethyl vanillin in a vanillin/ethyl vanillin molar ratio of 2, characterized in that it comprises an operation of cogranulation of vanillin and ethyl vanillin used in powder form and in amounts such that the vanillin/ethyl vanillin molar ratio is at least equal to 2, carried out at a temperature of between 50° C. and 57° C., followed by an operation enabling the temperature of the composition obtained to be brought back to ambient temperature.

In the present text, the expression “composition comprising essentially a compound based on vanillin and ethyl vanillin” is intended to mean a composition comprising at least 80% by weight of a mixture of the new vanillin/ethyl vanillin compound with a vanillin/ethyl vanillin molar ratio of 2 and of vanillin: the vanillin representing less than 25% by weight of said mixture.

The expression “new vanillin/ethyl vanillin compound” is intended to mean the compound in anhydrous form and hydrates thereof.

The term “cogranulation” is intended to mean an operation which consists, starting from vanillin and ethyl vanillin powders, in obtaining the new compound of the invention, in granule form.

The term “granulation” is intended to mean the forming of a powder in granule form.

In accordance with the invention, it has been found that the new compound of vanillin and ethyl vanillin is readily obtained according to this cogranulation process.

The applicant has found that the presence of an excess of vanillin can act as crystallization seeds and thus facilitate the crystallization of the new compound.

In order to ensure an excess of vanillin relative to the molar ratio of 2, the vanillin and the ethyl vanillin are used in the following proportions:
from 65 to 72% by weight of vanillin,
from 35 to 28% by weight of ethyl vanillin.

In accordance with a preferred mode of the invention in which a small excess of vanillin is preferred, the proportions are advantageously the following:
from 67 to 70% by weight of vanillin,
from 30 to 33% by weight of ethyl vanillin.

According to the first step of the process of the invention, homogeneous mixing of the vanillin and ethyl vanillin powders is first carried out.

To this effect, said powders are charged separately or as a mixture to a mixer-granulator, which is subjected to stirring.

Preferably, the stirring conditions are chosen such that there are no high shear forces.

Thus, a slow stirring speed is preferred.

By way of indication, it may be specified that, in the case of a mixer of plow type, the stirring conditions advantageously range between 0.2 and 1 m/s, at the end of the blades.

The mixture of powders is then brought to a temperature referred to in the remainder of the text as the “cogranulation temperature”.

This temperature is defined as being less than the melting point of the new compound of vanillin and ethyl vanillin which, measured by differential scanning calorimetry, is 60° C.±2° C.

Thus, the cogranulation temperature is advantageously chosen between 50 and 57° C., preferably between 50 and 55° C.

In accordance with the process of the invention, the mixture of vanillin and ethyl vanillin powders is brought from ambient temperature to the cogranulation temperature which is chosen as previously described.

The term “ambient temperature” is intended to mean generally a temperature between 15 and 25° C.

The rise in temperature is preferentially carried out gradually, for example 1° C. every 3 minutes.

Once the cogranulation temperature has been reached, the mixture is kept stirring at this temperature for a period of time sufficient to obtain the conversion of the reagents into an expected new compound.

The duration of this isothermal hold is determined as a function of the cogranulation temperature chosen.

The higher the chosen cogranulation temperature, the shorter the duration of the isothermal hold.

For example, for a cogranulation temperature advantageously chosen at 51° C., the duration of the hold advantageously ranges between 5 min and 1 hour, and preferably between 20 min and 40 min. It should be noted that the upper limit is not critical, but for productivity reasons, a duration at most equal to 1 hour is preferentially chosen.

For a temperature greater than or equal to 54° C., it is found that it is no longer necessary to maintain an isothermal hold. In other words, there is no longer any reason to keep the mixture stirring once the temperature has been reached.

It should be noted that the operations previously described are preferentially carried out under an atmosphere of inert gases, most commonly nitrogen.

In a subsequent step, the composition obtained is cooled to a temperature of less than 40° C.

According to one preferred embodiment, the composition is allowed to cool with stirring and under an inert atmosphere to a temperature of less than 40° C., preferably to a temperature of less than 35° C. The lower limit of the cooling temperature is advantageously ambient temperature.

A composition comprising the new vanillin/ethyl vanillin compound is recovered.

The various operations of the process of the invention can be carried out in a mixer which is advantageously a plow mixer or a ribbon mixer.

This mixer is advantageously fitted with a double jacket in order to provide the various heat transfers by circulation of a heat-transfer fluid in the double jacket. The heat-transfer fluid may be water kept at a temperature that is for example from 2 to 5° C. above the cogranulation temperature chosen, or any other heat-transfer fluid, for example a silicone oil.

In the case of cooling, the temperature of the heat-transfer fluid, in this case water, is generally chosen at a temperature which is for example from 2 to 5° C. below the cooling temperature chosen.

The compound obtained according to the process of the invention comprises at least 80% by weight, preferably at least 90% by weight of a mixture of the new vanillin/ethyl vanillin compound and of vanillin.

The composition obtained comprises less than 20% by weight, preferably less than 10% by weight of other crystalline phases of the vanillin/ethyl vanillin phase diagram and optionally of vanillin: this mixture subsequently being denoted “other crystalline phases”.

More specifically, the compositions obtained may comprise:
from 80 to 99% by weight of a mixture of the new vanillin/ethyl vanillin compound and of vanillin,
from 1 to 20% by weight of other crystalline phases.

The preferred compositions of the invention comprise:
from 90 to 99% by weight of a mixture of the new vanillin/ethyl vanillin compound and of vanillin,
from 1 to 10% by weight of other crystalline phases.

In the mixture obtained which comprises the new vanillin/ethyl vanillin compound and vanillin, the vanillin represents less than 20% by weight, preferably less than 14% by weight of said mixture.

More specifically, the mixtures obtained may comprise:
from 80 to 94% by weight of the new vanillin/ethyl vanillin compound,
from 6 to 20% by weight of vanillin.

The preferred mixtures have the following composition:
from 86 to 94% by weight of the new vanillin/ethyl vanillin compound,
from 6 to 14% by weight of vanillin.

The composition obtained is in the form of granules, the size of which ranges, for example, between 200 μm and 10 000 μm and preferably between 500 μm and 1000 μm.

In order for the size of the particles to be compatible with the application envisioned, a milling operation can be envisioned.

This operation is carried out in a such a way that the particle size, expressed by the median diameter (d₅₀), ranges from 200 μm to 1000 μm, and is preferably between 500 μm and 800 μm. The median diameter is defined as being such that 50% by weight of the particles have a diameter greater than or less than the median diameter.

The milling operation can be carried out in a conventional apparatus, such as a blade mill, a toothed roll crusher or a granulator.

The X-ray diffraction spectrum of the composition obtained has the lines at angles 2θ (°)=20.7-25.6-27.5-28.0 that are characteristic of the new compound of the invention.

With regard to its flowability properties, the composition of the invention has a flowability index after 24 hours of storage at 40° C. under air at 80% relative humidity, at a normal stress of 2 400 Pa, ranging between 0.05 and 0.6.

According to one preferred variant of the process of the invention, it has been found that it was particularly advantageous to carry out the first step of mixing the powders under a wet nitrogen atmosphere, thus resulting in a whiter product.

Thus, a small amount of water may be present in the nitrogen. It may represent from 1 to 5% of the weight of nitrogen, preferably from 2 to 3% of the weight of nitrogen.

The humidifying of the nitrogen stream can be carried out by sparging into water.

According to one preferred embodiment of the process of the invention, the mixing of the powders is begun under wet nitrogen, and then the temperature is gradually increased and, when the latter is greater than or equal to 44° C. and less than 49° C., dry nitrogen is introduced.

The “dry nitrogen” is intended to mean a stream of nitrogen comprising less than 0.5 g, preferably less than 0.3 g of water per kg of nitrogen.

In a subsequent step, the temperature of the composition obtained is brought back to ambient temperature as previously described.

The composition obtained has an X-ray diffraction spectrum which comprises the characteristic lines as illustrated by FIG. 1.

According to this variant of implementation under wet nitrogen, a composition which is whiter in color is obtained more rapidly since the isothermal hold can be shortened. For example, following a rise in temperature under dry nitrogen, an isothermal hold at 52° C. for a period of 2 hours is desirable. If the rise in temperature is carried out under wet nitrogen, an isothermal hold at 52° C. for a period of 30 minutes is sufficient.

The process of the invention applies to vanillin and ethyl vanillin produced by any chemical synthesis, regardless of the starting substrate.

It is also suitable for vanillin obtained according to biochemical processes, in particular processes of microbiological fermentation, especially ferulic acid.

The invention does not exclude the use of an excipient or excipients with the composition of the invention.

It should be noted that the choice of the excipient(s) must take into account the intended use of the final product and therefore it must be edible if it is used in the food sector.

The amount of excipient(s) can be very variable and it can represent from 0.1 to 90% of the weight of the final mixture.

It is advantageously selected between 20 and 70% by weight.

Depending on the type of excipient selected, the amount used and the intended use of the final product, the excipient can be introduced, wholly or partly, at the end of the preparation of the composition of the invention or during the preparation of the composition of the invention. In other words, the total amount of the excipient(s) can be introduced during the preparation of the composition of the invention or else added at the end of the preparation of the composition of the invention. It is also possible to fractionate the amounts used during the preparation or after the preparation.

It may be specified, by way of example, that it is possible to add from 5 to 50% by weight of an excipient during the preparation of the composition of the invention and then to again add from 5 to 50% by weight of said excipient when the preparation of the composition of the invention is finished.

It is also possible to modulate the types of introduction depending on the excipients, i.e. to introduce the total amount of an excipient for example during the preparation of the composition of the invention and to fractionate the amount added of another excipient, or vice versa.

According to a first variant, the excipient is added by dry mixing with the obtained composition of the invention.

According to another variant, the excipient can be incorporated into the process for obtaining the composition of the invention, for example during the step of cogranulation of the vanillin and ethyl vanillin mixture.

It goes without saying that the same excipient can be added, fractionated, at these two stages of production or that excipients of different nature can also be introduced during or at the end of the preparation of the composition of the invention.

Examples of excipients that can be used are given hereinafter, but are given without being limiting in nature.

Fatty substances represent a first type of excipient.

As examples, mention may be made of fatty acids optionally in the form of salts or esters.

The fatty acids used are generally long-chain saturated fatty acids, i.e. fatty acids having a chain length between approximately 9 and 21 carbon atoms, such as, for example, capric acid, lauric acid, tridecylic acid, myristic acid, palmitic acid, stearic acid or behenic acid.

It is possible for said acids to be in salified form and mention may in particular be made of calcium stearate or magnesium stearate.

As fatty acid esters, mention may in particular be made of glyceryl stearate, isopropyl palmitate, cetyl palmitate and isopropyl myristate.

Mention may also more specifically be made of esters of glycerol and of long-chain fatty acids, such as glyceryl monostearate, glyceryl monopalmitostearate, glyceryl palmitostearate, ethylene glycol palmitostearate, polyglyceryl palmitostearate, polyglycol 1500 and 6000 palmitostearate, glyceryl monolinoleate; optionally mono- or diacetylated glycerol esters of long-chain fatty acids, such as monoacetylated or diacetylated monoglycerides and mixtures thereof; semisynthetic glycerides.

It is also possible to add a fatty alcohol of which the chain of carbon atoms is between approximately 16 and 22 carbon atoms, such as, for example, myristyl alcohol, palmityl alcohol or stearyl alcohol.

It is also possible to use polyoxyethylenated fatty alcohols resulting from the condensation of linear or branched fatty alcohols, having from 10 to 20 carbon atoms, with ethylene oxide in a proportion from 6 to 20 mol of ethylene oxide per mole, such as, for example, coconut alcohol, tridecanol or myristyl alcohol.

Mention may also be made of waxes such as microcrystalline waxes, white wax, carnauba wax or paraffin.

Mention may be made of sugars, for instance glucose, sucrose, fructose, galactose, ribose, maltose, sorbitol, mannitol, xylitol, lactitol, maltitol; invert sugars: glucose syrups and also sucroglycerides derived from fatty oils such as coconut oil, palm oil, hydrogenated palm oil and hydrogenated soybean oil; sucrose esters of fatty acids, such as sucrose monopalmitate, sucrose monodistearate and sucrose distearate.

As examples of other excipients, mention may be made of polysaccharides, and mention may be made, inter alia, of the following products and mixtures thereof:
native, pregelatinized or modified starches derived in particular from wheat, corn, barley, rice, cassava or potato, and more particularly native corn starches rich in amylose, pregelatinized corn starches, modified corn starches, modified waxy corn starches, pregelatinized waxy corn starches, modified waxy corn starches, in particular the OSSA/sodium octenylsuccinate starch,
starch hydrolysates,
dextrins and maltodextrins resulting from the hydrolysis of a starch (wheat, corn) or of a potato flour, and also β-cyclodextrins,
cellulose, ethers thereof, in particular methyl cellulose, ethyl cellulose, methylethyl cellulose, hydroxypropyl cellulose; or esters thereof, in particular carboxymethyl cellulose or carboxyethyl cellulose optionally in sodium-containing form,
gums, such as carrageenan gum, Kappa-carrageenan or Iota-carrageenan gum, pectin, guar gum, locust bean gum, xanthan gum, alginates, gum arabic, acacia gum, agar-agar.

A maltrodextrin having a degree of hydrolysis measured by “dextrose equivalent”, or DE, of less than 20 and preferably between 5 and 19, and more preferentially between 6 and 15, is preferentially selected.

As other excipients, mention may be made of flours, in particular wheat flour (native or pregel); starches, more particularly potato flour, arrowroot starch, corn starch, cornflour, sago or tapioca.

By way of excipients, use may also be made of gelatin (preferably having a gelling strength using a gelometer of 100, 175 and 250 Bloom). It can without distinction come either from acid treatment of pig skin and ossein, or from alkaline treatment of cowhide and ossein.

It is also possible to add other excipients, such as silica or else, for example, an antioxidant such as, in particular, vitamin E or an emulsifier, in particular lecithin.

In order to adjust the flavoring power of the mixture or enhance its taste, the use of ethylmaltol and/or of propenyl guaethol can be envisioned.

The invention does not exclude the addition of a supplementary amount of vanillin or ethyl vanillin.

The choice of the excipients is made as previously mentioned according to the application envisioned.

The composition of the invention can also be used in many fields of application, inter alia, in the food and pharmaceutical sector, and in the perfumery industry.

A preferred field of application of the use of the composition of the invention is in the cookie trade and cake-making industry, and more particularly:
dry cookie trade: sweet cookies of conventional type, butter cookies, large round cookies, snacks, shortbread,
factory-baked cakes: champagne ladyfingers, thin fingers, sponge fingers, Genoa cake, sponge cake, madeleines, pound cakes, fruit cakes, almond cakes, petit fours.

The essential elements present in the mixtures intended for the abovementioned industries are proteins (gluten) and starch, which are most commonly provided by wheat flour. For preparing the various types of cookies and cakes, ingredients such as sucrose, salt, eggs, milk, fat, optionally chemical yeasts (sodium bicarbonate or other artificial yeasts) or biological yeasts and flours from various cereals, etc. are added to the flour.

The composition according to the invention is incorporated during the manufacture, depending on the desired product, according to conventional techniques in the field under consideration (cf. in particular J. L. Kiger and J. C. Kiger—Techniques Modernes de la Biscuiterie, Pâtisserie-Boulangerie industrielles et artisanales [Modern techniques of industrial and traditional production of cookies, cakes and bakery products], DUNOD, Paris, 1968, Volume 2, pp. 231 ff.).

Preferentially, the composition of the invention is introduced into the fats which are used in the preparation of the dough.

By way of indication, it will be specified that the composition of the invention is introduced in an amount of from 0.005 to 0.2 g per kg of dough.

The composition of the invention is perfectly suitable for use in the chocolate-making field, regardless of the form in which it is used: bars of chocolate, couverture chocolates, filling for chocolates.

It can be introduced during conching, i.e. blending of the cocoa paste with the various ingredients, in particular flavorings, or after conching, by processing in the cocoa butter.

In this field of application, the composition of the invention is used, depending on the type of chocolate, in a proportion of from 0.0005 g to 0.1 g per 1 kg of final product: the highest contents being used in couverture chocolate.

Another use of the composition of the invention is the manufacture of candies of all kinds: sugared almonds, caramels, nougats, hard candies, fondant candies and the like.

The amount of the composition of the invention introduced depends on the more or less strong taste that is desired. Thus, the doses of use can range between 0.001% and 0.2%.

The composition of the invention is very suitable for uses in the dairy industry, and more particularly in flavored and gelled milks, cream desserts, yoghurts, ices and ice creams.

The flavoring is carried out by simple addition of the composition of the invention, in one of the mixing stages required during production of the product.

The contents of said composition to be used are generally low, about 0.02 g per 1 kg of final product.

Another application of the composition of the invention in the food sector is the preparation of vanillin sugar, i.e. impregnation of sugar with vanillin, in a content of about 7 g expressed relative to 1 kg of final product.

The composition of the invention can also be included in various drinks, and mention may be made, inter alia, of grenadine and chocolate drinks.

In particular, it can be used in preparations for instant drinks delivered by automatic drinks dispensers, flavored drinks powders, chocolate powder or else in instant preparations in the form of powder intended for making desserts of all kinds, custard tarts, cake mixtures, pancakes, after dilution with water or with milk.

It is common practice to use vanillin for denaturing butter. To this effect, the composition of the invention can be used in a proportion of 6 g per metric tonne of butter.

Another field of application of the composition of the invention is animal feed, in particular for preparing meal for calf and pig feeds. The recommended content is approximately 0.2 g per kg of meal to be flavored.

The composition of the invention can find other applications, such as a masking agent, for the pharmaceutical industry (for masking the odor of a medicament) or for other industrial products (such as gum, plastic, rubber, etc.).

It is entirely suitable in completely different fields such as the cosmetics industry, the perfumery industry or the detergent industry.

It can be used in cosmetics such as creams, milks, make-up and other products, and also, as fragrancing ingredients, in fragrancing compositions and fragranced substances and products.

The term “fragrancing compositions” denotes mixtures of various ingredients such as solvents, solid or liquid carriers, fixing agents, various odorous compounds, etc., into which the composition of the invention is incorporated and is used to give the desired fragrance to various types of final product.

Fragrance bases constitute preferred examples of the fragrancing compositions in which the composition of the invention can advantageously be used at a content of from 0.1% to 2.5% by weight.

The fragrance bases can be used for preparing numerous fragranced products, such as, for example, eaux de toilettes [toilet waters], fragrances, aftershave lotions; toiletries and hygiene products, such as bath or shower gels, deodorant or antiperspirant products, whether in the form of sticks or lotions, talcs or powders of any nature; products for the hair, such as shampoos and hair products of any type.

Another example of use of the composition of the invention is the soap-making field. It can be used in a content of from 0.3% to 0.75% of the total mass to be fragranced. Generally, it is combined, in this application, with benzoin resinoid and sodium hyposulfite (2%).

The composition according to the invention can find many other applications, in particular in air fresheners or any maintenance product.

The physicochemical characteristics of the compositions of the invention are determined according to the following methods:

### 1. Melting Point

The melting point of the composition of the invention is measured by differential scanning calorimetry.

The measurement is carried out using a Mettler DSC822e differential scanning calorimeter under the following conditions:
preparation of the sample at ambient temperature: weighing out and introduction into a sample carrier,
sample carrier: crimped aluminum capsule,
test specimen: 8.4 mg,
rate of temperature increase: 2° C./min,
study range: 10-90° C.

The sample of the composition is weighed out and introduced into the capsule, which is crimped and then placed in the apparatus.

The temperature program is run and the melting profile is obtained on a thermogram.

The melting temperature is defined on the basis of a thermogram produced under the above operating conditions.

The onset temperature is retained: temperature corresponding to the maximum slope of the melting peak.

### 2. X-ray Diffraction Spectrum

The X-ray diffraction spectrum of the composition of the invention is determined using the X'Pert Pro MPD PANalytical apparatus equipped with an X' Celerator detector, under the following conditions:
Start Position [°2Th.]: 1.5124
End Position [°2Th.]: 49.9794
Step Size [°2Th.]: 0.0170
Scan Step Time [s]: 41.0051
Anode Material: Cu
K-Alphal [Å]: 1.54060
Generator Settings: 30 mA, 40 kV

### 3. Flowability Property and Caking Index

The composition of the invention has the characteristic of caking less on storage, which is demonstrated by determining the flowability index of the powder.

The flowability of powders is a technical notion well known to those skilled in the art. For further details, reference may be made in particular to the handbook “Standard shear testing technique for particulate solids using the Jenike shear cell”, published by “The Institution of Chemical Engineers”, 1989 (ISBN: 0 85295 232 5).

The flowability index is measured in the following way.

The flowability of powders is measured by shearing a sample in an annular cell (sold by D. Schulze, Germany).

The preshearing of the powders is carried out under a normal stress of 5200 Pa.

The shear points necessary for plotting the yield locus of the sample are obtained for four normal stresses below the stress of the preshearing, typically 480 Pa, 850 Pa, 2050 Pa and 3020 Pa.

From the Mohr circles in the diagram of “shear stress as a function of normal stresses”, two stresses are determined on the yield locus, which stresses characterize the sample:
the normal stress in the main direction; it is given by the end of the large Mohr circle which passes through the preshear point,
the cohesive force; it is given by the end of the small Mohr circle which is tangent to the yield locus and passes through the origin.

The ratio of the normal stress in the main direction to the cohesive force is a dimensionless number, referred to as “i, flowability index”.

These measurements are carried out immediately after filling the annular cell; the immediate flowability index is thus obtained.

Another series of measurements is carried out with a cell which has been stored for 24 hours at 40° C. and 80% relative humidity under a normal stress of 2400 Pa.

The caking index is thus obtained.

Examples illustrating the present invention, without being limiting in nature, are given hereinafter.

In the examples, the percentages mentioned are expressed by weight.

### EXAMPLE 1

2100 g of powdered vanillin (VA) and 900 g of ethyl vanillin (EVA), i.e. a VA/EVA weight ratio=70/30, are introduced into a plow mixer equipped with a tank that has a volume of 15 liters and heated by means of a double jacket. The moisture content of these powders is 0.1% by weight.

The stirring is started at the speed of 20 rpm, i.e. a blade-end speed of 0.25 m/s. This stirring speed is kept constant throughout all the phases of the process.

Circulation of wet nitrogen is established in the mixer with a flow rate of 200 l/h. The humidifying of the nitrogen stream is carried out by sparging into water kept at 40° C. so as to obtain 25 g of water per kg of nitrogen. The feed line between the water bath and the mixer is kept at 45° C. so as to prevent any condensation in the pipes.

The temperature of the heat-transfer fluid circulating in the double jacket is gradually increased in such a way that the temperature of the mixture of powders follows a ramp of +0.3° C./min.

When the temperature of the product reaches 49.5° C., the water bath humidifying the nitrogen stream is bypassed so as to feed the mixer with a dry nitrogen circulation (less than 0.5 g of water/kg of nitrogen). At the same time, 15 g of Tixosil 365 silica are introduced into the mixer.

The temperature of the product is brought from 49.5° C. to 52° C. at +0.2° C./min and then is kept at 52° C. for 30 minutes. The heating of the heat-transfer fluid is then stopped and the temperature of the product is brought back to 30° C. by natural cooling. The stirring and the nitrogen circulation are stopped. The mixer is drained off.

The product is screened at 800 μm; the material passing through represents 56% by weight of the total weight. The oversize at 800 μm is milled using a Quadro Comill mill fitted with an 800 μm screen. The 2 fractions are then combined and the mixture is homogenized to give the final product.

The melting point of the granules is determined by differential scanning calorimetry as previously described. The thermogram obtained shows a main peak which corresponds to the new vanillin/ethyl vanillin compound. The melting temperature (Tonset) which corresponds to the maximum slope of the peak is 59.5° C.

The X-ray diffraction spectrum of the granules exhibits the characteristic lines at angles 2θ=20.7-25.6-27.5-28.0, as shown in FIG. 1, and which distinguish it from the vanillin and ethyl vanillin spectra.

The flowability index and the caking index, measured as described previously using an annular cell, are respectively 5.70 and 0.09.

### EXAMPLE 2

The procedure of example 1 is repeated with the following modifications only:
a stirring speed of 40 rpm,
a temperature increase ramp under wet nitrogen of +0.5° C./min,
a final temperature under dry nitrogen of 55° C.,
no isothermal hold before cooling.

The increase in the final cogranulation temperature makes it possible to eliminate the isothermal hold, guaranteeing complete conversion of the vanillin/ethyl vanillin mixture into the new compound. On the other hand, the increase in size of the granules is greater, since, at the mixer outlet, the material passing through at 800 μm now represents only 27% of the total weight, which makes it necessary to mill 73% of the product.

After milling of the oversize at 800 μm and mixing of the 2 fractions, the product obtained has a flowability index of 6.30 and a caking index of 0.10.

### EXAMPLE 3

The procedure of example 1 is repeated, with the only difference being that the 15 g of Tixosil 365 silica are replaced with 150 g of Roquette IT12 maltodextrin.

At the mixer outlet, the material passing through at 800 μm represents 55% of the total weight.

After milling of the oversize at 800 μm and mixing of the 2 fractions, the product obtained has a flowability index of 5.90 and a caking index of 0.12.

### EXAMPLE 4

In this example, a composition is prepared in the form of granules comprising 50% by weight of the granules prepared according to example 1 and 50% by weight of Roquette IT6 maltodextrin.

The mixing operation, which lasts approximately 5 min, is carried out at ambient temperature under ambient air atmosphere in the plow mixer with a rotational speed of 60 rpm.

The mixture thus obtained has a flowability index of 8.80 and a caking index of 0.62.

Its aromatic power is equivalent to that of pure vanillin.

1. A process for preparing a composition, the process comprising cogranulating vanillin and ethyl vanillin in powder form and in amounts such that a resulting vanillin/ethyl vanillin molar ratio is at least equal to 2, carried out at a temperature of from 50° C. to 57° C., followed by bringing the temperature of the composition obtained back to ambient temperature.

2. The process as defined by claim 1, wherein the vanillin and the ethyl vanillin are used in the following proportions:
from 65% to 72% by weight of vanillin, and
from 28% to 35% by weight of ethyl vanillin.

3. The process as as defined by claim 1, wherein the vanillin and the ethyl vanillin are used in the following proportions:
from 67% to 70% by weight of vanillin, and
from 30% to 33% by weight of ethyl vanillin.

4. The process as defined by claim 1, wherein the mixing of the vanillin and ethyl vanillin powders is carried out with stirring, and that the mixture is brought from ambient temperature to a cogranulation temperature.

5. The process as defined by claim 4, wherein the cogranulation temperature is from 50° C. to 55° C.

6. The process as defined by claim 4, wherein the increase in temperature is carried out gradually.

7. The process as defined by claim 4, wherein once the cogranulation temperature has been reached, the mixture is kept stirring at this temperature for a period of time sufficient to obtain conversion of the reagents into an expected new compound.

8. The process as defined by claim 1, wherein the operations are carried out under an atmosphere of an inert gas.

9. The process as defined by claim 1, wherein mixing is begun under wet nitrogen, and then the temperature is gradually increased and, when the temperature is greater than or equal to 44° C. and less than 49° C., dry nitrogen is introduced.

10. The process as defined by claim 9, wherein the nitrogen stream comprises from 1% to 5% by weight of water.

11. The process as defined by claim 1, wherein the composition obtained is cooled with stirring and under an inert atmosphere to a temperature of less than 40° C.

12. The process as defined by claim 1, wherein the composition obtained is formed according to a milling technique.

13. The process as defined by claim 1, wherein an excipient or excipients is (are) added to the composition.

14. The process as defined by claim 1, wherein the excipient(s) is (are) added wholly or partly by dry mixing with the composition previously obtained or during the production of said composition.

15. A composition as defined by claim 1, wherein the composition comprises:
from 80% to 99% by weight of a mixture of the new vanillin/ethyl vanillin compound and of vanillin, and
from 1% to 20%, by weight of other crystalline phases.

16. The composition as defined by claim 15, wherein the mixture obtained comprises:
from 80% to 94% by weight of the new vanillin/ethyl vanillin compound, and
from 6% to 20% by weight of vanillin.

17. A composition comprising at least one composition as defined by claim 15, and at least one excipient selected from the group consisting of a fatty substance; a fatty alcohol; a sugar; a polysaccharide; a silica; a vanillin and an ethyl vanillin.

18. The composition as defined by claim 17, wherein the excipient is selected from the group consisting of:
a sugar an invert sugar: a glucose syrup, and also a sucroglyceride derived from a fatty oil; and a sucrose ester of a fatty acid,
a native, pregelatinized or modified starch optionally derived from wheat, corn, barley, rice, cassava or potato, a native corn starch rich in amylose, a pregelatinized corn starch, a modified corn starch, a modified waxy corn starch, a pregelatinized waxy corn starch, a modified waxy corn starch, optionally a OSSA/sodium octenylsuccinate starch,
a starch hydrolysate,
a dextrin and a maltodextrin resulting from the hydrolysis of a starch (wheat, corn) or of a potato flour, and also a β-cyclodextrin, optionally a maltrodextrin having a DE of less than 20,
cellulose, ethers thereof, optionally methyl cellulose, ethyl cellulose, methylethyl cellulose, hydroxypropyl cellulose; or an ester thereof, optionally carboxymethyl cellulose or carboxyethyl cellulose optionally in sodium-containing form,
a gum,
a flour; a starch,
gelatin,
silica,
an antioxidant,
an emulsifier, and
vanillin or ethyl vanillin.

19. The composition as defined by claim 17, wherein the composition comprises from 0.1% to 90% by weight of excipient(s).

20. A method of making a flavorant or fragrance, the method comprising making the flavorant or fragrance using an effective amount of the composition as defined by claim 15, wherein the flavorant or fragrance can be used in human food or animal feed, in a pharmaceutical, in a cosmetic, in a perfume or in a detergent.

21. The method as defined by claim 20, wherein the flavorant or fragrance is used during manufacture of a dough, optionally in a fat, in the field of the dry cookie trade and factory-baked cakes; in the chocolate-making field, optionally for the preparation of bars of chocolate, couverture chocolates or filling for chocolates; during manufacture of candies including sugared almonds, caramels, nougats, hard candies, fondant candies; in the dairy industry and optionally in flavored and gelled milks, cream desserts, yoghurts, ices and ice creams; in the preparation of vanillin sugar, by impregnation of sugar with vanillin; in the preparation of various drinks, optionally grenadine and chocolate drinks; in the preparation of instant drinks including flavored drinks powders, chocolate powder or else in instant preparations in the form of powder intended for making desserts; for denaturing butter.

22. The method as defined by claim 20, wherein the flavorant or fragrance is used in animal feed, optionally for preparing meal.

23. The method as defined by claim 20, wherein the flavorant or fragrance is used as an odor-masking agent, in the pharmaceutical industry; in cosmetics industry for preparing creams, milks and make-up and other products or in the detergent industry, optionally in soap-making.

24. The process as defined by claim 8, wherein the inert gas is nitrogen.

25. The process as defined by claim 10, wherein the nitrogen stream comprises from 2% to 3% by weight of water.

26. The process as defined by claim 11, wherein the temperature is less than 35° C.

27. The process as defined by claim 14, wherein the excipients are added during the cogranulation of the vanillin and ethyl vanillin.

28. The composition as defined by claim 15, wherein the composition comprises from 80% to 94% by weight of the mixture of the new vanillin/ethyl vanillin compound and of vanillin.

29. The composition as defined by claim 15, wherein the composition comprises from 1% to 10% by weight of other crystalline phases.

30. The composition as defined by claim 16, wherein the composition comprises from 86% to 94% by weight of the new vanillin/ethyl vanillin compound.

31. The composition as defined by claim 16, wherein the composition comprises from 6% to 14% by weight of vanillin.

32. The composition as defined by claim 18, wherein the sugar is selected from the group consisting of glucose, sucrose, fructose, galactose, ribose, maltose, sorbitol, mannitol, xylitol, lactitol, and maltitol.

33. The composition as defined by claim 18, wherein the sucroglyceride derived from a fatty oil is selected from the group consisting of coconut oil, palm oil, hydrogenated palm oil and hydrogenated soybean oil.

34. The composition as defined by claim 18, wherein the sucrose ester of a fatty acid is selected from the group consisting of sucrose monopalmitate, sucrose monodistearate and sucrose distearate.

35. The composition as defined by claim 18, wherein the gum is selected from the group consisting of a carrageenan gum, a Kappa-carrageenan or Iota-carrageenan gum, a pectin, a guar gum, a locust bean gum, a xanthan gum, an alginate, a gum arabic, an acacia gum, and agar-agar.

36. The composition as defined by claim 18, wherein the antioxidant is vitamin E.

37. The composition as defined by claim 18, wherein the emulsifier is lecithin.

38. The composition as defined by claim 19, wherein the composition comprises from 20% to 70% by weight of excipient(s).
